(19) United States
(12) Patent Application Publication
(10) Pub. No.: US 2003/0022347 A1
(43) Pub. Date: Jan. 30, 2003

(54) Bacterial galactanases and use thereof

(75) Inventors: Carsten Sjoholm, Allerod (DK); Peter Rahbek Ostergaard, Virum (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 09/777,597

(22) Filed: Feb. 6, 2001

Related U.S. Application Data
(60) Provisional application No. 60/181,919, filed on Feb. 11, 2000.

(30) Foreign Application Priority Data
Feb. 8, 2000 (DK) PA 2000 00196
Feb. 10, 2000 (DK) PA 2000 00212

(51) Int. Cl. C12N 9/40; C12P 21/02; C12N 1/21; C07H 21/04; C12N 15/74

(52) U.S. Cl. 435/208; 435/69.1; 435/252.31; 435/320.1; 536/23.2

(57) ABSTRACT

The invention relates to an isolated polypeptide with galactanase activity, polynucleotides encoding a galactanase, a method for modifying animal feed, in particular animal feed comprising plant material such as soybean, by adding to the animal feed at least one galactanase enzyme, and also to a method for obtaining a DNA sequence encoding a galactanase enzyme or a portion thereof.

### CROSS-REFERENCE TO RELATED APPLICATIONS

This application claims priority under 35 U.S.C. 119 of Danish application nos. PA 2000 00196 filed Feb. 8, 2000 and PA 2000 00212 filed Feb. 10, 2000, and of U.S. provisional application No. 60/181,919 filed Feb. 11, 2000, the contents of which are fully incorporated herein by reference.

### FIELD OF THE INVENTION

The present invention relates to bacterial galactanase enzymes for use in different industrial applications, such as in the textile, detergent and cellulose fiber processing industries, and in particular to methods for modifying and/or improving the nutritional value of an animal feed using such enzymes.

### BACKGROUND OF THE INVENTION

Galactans and arabinogalactans are present in most plants as components of pectic hairy regions and can be found in high quantities e.g. in soy plant seed and in potatoes. Another good source for highly purified galactans and arabinogalactans is the water-soluble polysaccharide extracted with alkali from lupin fibre. This substrate can be treated with arabinofuranosidase (EC 3.2.1.55) resulting in a galactan with a very high content of galactose (more than 91%); such a substrate can be obtained from Megazyme, Ireland.

Galactans and arabinogalactans are usually attached to O-4 of rhamnose residues in the rhamnogalacturonan backbone of the hairy region. The distribution and composition of the sidechains vary considerably between different cell types and physiological states, but in general about half of the rhamnosyl units in the rhamnogalacturonan regions have sidechains attached. The galactan sidechains are in most plants type 1 galactans, which are composed of β-1,4 linked galactopyranose with some branching points and a length of up to 60 saccharide units (DP60). Arabinofuranose residues or short arabinan oligomers can be attached to the galactan chain at the O-3 of the galactosyl unit, thus the name arabinogalactan. Galactans (or arabinogalactans) have an important function in the primary cell wall, where they interact with other structural components of the cell wall such as xyloglucans or arabinoxylans. Thus, they possibly serve to anchor the pectic matrix in the cell wall. Furthermore, they increase the hydration and water-binding capacity and decrease inter-chain association between pectin polymers, which is thought to be of importance for modulation of porosity and passive diffusion. Beta-1,4-galactanases (EC 3.2.1.89) degrade galactans (and arabinogalactans) and have been purified from a variety of microbial sources.

WO 92/13945 describes cloning and DNA sequencing of a fungal beta-1,4-galactanase from Aspergillus aculeatus.

WO 97/32014 describes cloning and DNA sequencing of fungal beta-1,4-galactanase from Humicola insolens and Myceliophthora thermophilum.

WO 97/32013 describes cloning and DNA sequencing of fungal beta-1,4-galactanase from Meripilus giganteus.

Braithwaite et al., BIOCHEMISTRY Vol. 36, No. 49 pp. 15489-15500 (1997) disclose a galactanase from Pseudomonas fluorescens ssp. cellulosa which is a retaining family 53 glycosyl hydrolase in which e161 and e270 are the catalytic residues.

WO 91/18521 describes a feed composition comprising, as a source of carbohydrates, a mannan-containing hemicellulose selected from soybeans, corn and alfalfa, as well as a mannanase that catalyzes the degradation of the mannan-containing hemicellulose. WO 97/16982 and WO 00/47711 describe Bacillus galactanases and use of galactanases in animal feed.

Nakano et al., Eur. J. Biochem. 193(1): 61-67 (1990) describes the purification and characterization of an exo-1,4-β-galactanase from a strain of Bacillus subtilis.

The entries from the publicly available databases listed below in Table 1 refer to sequences with homology to the galactanases described herein:

TABLE 1
Columns specify the accession numbers in the different databases.

| Species | EMBL | GeneSeq Nucl | Swissprot | GeneSeq Prot. |
|---|---|---|---|---|
| A_aculeatus * | L34599 | | P48842 | |
| B_agaradhaerens | | A59394 | | B07810 |
| B_circulans ** | L03425 | | P48843 | |
| B_licheniformis | | A59392 | | B07808 |
| B_subtilis ** | Z94043 | | | |
| H_insolens * | | T85059 | | W27064 |
| M_giganteus * | | T79497 | | W23140 |
| M_thermophila * | | T85058 | | W27063 |
| P_fluorescens * | X91885 | | P48841 | |

* The sequence is known in the public domain.
** The sequence is known in the public domain, but galactanase activity has not been reported.

In spite of the state of the art e.g. as disclosed above, there remains a need for galactanase enzymes with improved activity for a number of different purposes. The object of the present invention is to provide galactanase enzymes with a high galactanase activity for use in industrial applications, such as the textile, detergent and cellulose fiber processing industries, and in particular for the modification and/or improving the nutritional value of an animal feed.

### SUMMARY OF THE INVENTION

The problem to be solved by the present invention is to provide galactanases with improved properties for the use in the industry. The solution is based on that the inventors have isolated and identified a novel galactanase from the Bacillus genus, more specifically from a Bacillus pumilus.

Surprisingly only one conserved motif was found to be present in all Bacillus galactanases including the novel B. pumilus galactanase, this conserved motif comprises the amino acid sequence: Nxx(M/L)FDFxGxxLxS as shown in SEQ ID NO: 1, wherein x is any amino acid, and (M/L) is either M or L. As shown herein the galactanase of the B. pumilus has improved properties over the known bacterial galactanases, especially with respect to acid stability.

Accordingly in a first aspect the invention relates to an isolated polypeptide having galactanase activity comprising an amino acid sequence: Nxx(M/L)FDFxGxxLxS, as shown in SEQ ID NO: 1, wherein x may be any amino acid and (M/L) is M or L, and wherein the polypeptide maintains at least 30% residual galactanase activity after two hours at a temperature of 37° C. at pH 4.5, measured as defined herein.

The inventors have isolated and sequenced a polynucleotide molecule which encodes the novel galactanase of B. pumilus. The sequence of the polynucleotide molecule, which is a DNA sequence, is shown in SEQ ID NO: 2 and the amino acid sequence of the novel B. pumilus galactanase encoded by this DNA sequence is shown in SEQ ID NO: 3.

Accordingly in a second aspect the invention relates to an isolated polypeptide having galactanase activity comprising an amino acid sequence as shown in SEQ ID NO: 3 from residue 1 to residue 402; or comprising an amino acid sequence at least 70% identical, preferably 75%, or 80%, even more preferably 85%, or 90%, or most preferably 95% identical to the amino acid sequence of SEQ ID NO: 3 from amino acid residue 1 to amino acid residue 402.

A sequence alignment comparison was performed by the present inventors between the novel B. pumilus galactanase encoding polynucleotide and the ones known in the art, which showed that the highest degree of identity was as low as around 52% (See Table 3).

Consequently a third aspect of the invention relates to an isolated polynucleotide molecule encoding a polypeptide having galactanase activity comprising a polynucleotide sequence as shown in SEQ ID NO: 2 from position 1 to position 1209; or comprising a polynucleotide sequence at least 70%, preferably 75%, or 80%, even more preferably 85%, or 90%, or most preferably 95% identical to the polynucleotide sequence of SEQ ID NO: 2 from position 1 to position 1209.

The inventors isolated the polynucleotide sequence encoding the novel galactanase and cloned it in a vector, wherefrom the novel galactanase was expressed as described elsewhere herein. Various methods for manipulating and expressing a polynucleotide sequence are well known in the art, as likewise described herein. The polynucleotide sequences of the invention may be DNA, as described herein.

Hence a fourth aspect of the invention relates to an expression vector comprising a DNA segment selected from the group consisting of:

a) DNA molecules encoding a polypeptide having galactanase activity comprising a nucleotide sequence as shown in SEQ ID NO: 2 from nucleotide 1 to nucleotide 1209,

b) DNA molecules encoding a polypeptide having galactanase activity that is at least 70%, preferably 75%, or 80%, even more preferably 85%, or 90%, or most preferably 95% identical to the amino acid sequence of SEQ ID NO: 3 from amino acid residue 1 to amino acid residue 402,

c) DNA molecules encoding a galactanase enzyme comprising the amino acid sequence: Nxx(M/L)FDFxGxxLxS as shown in SEQ ID NO: 1, wherein x is any amino acid and (M/L) is M or L, and wherein the galactanase maintains at least 30% residual galactanase activity after two hours at a temperature of 37° C. at pH 4.5, measured as defined herein; and

d) degenerate DNA sequences of (a) (b) or (c).

Further a fifth aspect of the invention relates to a cell into which has been introduced an expression vector according to the previous aspect, wherein said cell expresses the polypeptide encoded by the DNA segment.

As a novel galactanase with improved properties like a galactanase of the invention is potentially very interesting for industrial purposes, processes or methods for the production of such a galactanase is also anticipated as being part of the invention. Such processes and methods are legio in the art, as mentioned herein.

Thus an aspect of the invention relates to a method of producing a polypeptide having galactanase activity comprising culturing a cell into which has been introduced an expression vector according to the fourth aspect, whereby said cell expresses a polypeptide encoded by the DNA segment; and recovering the polypeptide.

It is likewise envisioned to mix a galactanase of the invention with various enzymes in preparations or formulations in order to achieve synergistic effects in industrial applications.

Consequently an aspect of the invention relates to an enzyme preparation comprising an isolated polypeptide as defined in the first or second aspects.

In particular the inventors envision the industrial usage of a galactanase of the invention to be in methods for the modification and/or improving the nutritional value of an animal feed as also described elsewhere herein. The galactanase can be fed to the animal before, after, or simultaneously with the diet, or the galactanase can be used in a pre-treatment of the feed.

Accordingly another aspect of the invention is a method for modifying and/or improving the nutritional value of an animal feed, the method comprising adding to the animal feed at least one galactanase enzyme selected from the group consisting of:

a) a galactanase enzyme comprising an amino acid sequence as shown in SEQ ID NO: 3;

b) an analogue of the enzyme defined in a), said analogue comprising an amino acid sequence which is at least 70% identical to the sequence shown in SEQ ID NO: 3, or which can be derived from said sequence by substitution, deletion or addition of at least one amino acid; and

c) a galactanase enzyme comprising the amino acid sequence: Nxx(M/L)FDFxGxxLxS as shown in SEQ ID NO: 1, wherein x is any amino acid and (M/L) is M or L, and wherein said galactanase maintains at least 30% residual galactanase activity after two hours at a temperature of 37° C. at pH 4.5, measured as defined herein.

Yet another aspect of the invention relates to a method for modifying and/or improving the nutritional value of animal feed, the method comprising adding to the animal feed at least one galactanase enzyme comprising an amino acid sequence encoded by a DNA sequence which hybridizes to the DNA sequence shown in SEQ ID NO: 2 under at least medium stringency conditions, and preferably at high stringency conditions as defined herein.

As described herein the inventors identified several improved characteristics of a galacatanase of the invention when comparing it to known galactanases. A distinguishing feature of a galactanase of the invention is that it is encoded by a polynucleotide sequence which was found to have very little percentage identity with sequences encoding other galactanases (see Table 3). Methods are known in the art, as described herein whereby it is possible to use e.g. the information contained in a DNA sequence encoding a desirable polypeptide to obtain other DNA sequences encoding polypeptides with similar properties.

Accordingly an aspect of the invention relates to a method for obtaining a DNA sequence encoding a galactanase enzyme or a portion thereof, wherein said DNA sequence is detected using a probe comprising a DNA subsequence of the DNA sequence shown in SEQ ID NO: 2 or of a DNA sequence complementary to the DNA sequence shown in SEQ ID NO: 2, said subsequence comprising at least 16 nucleotides, e.g. at least 18 nucleotides.

Further an aspect of the invention relates to a method for obtaining a DNA sequence encoding a galactanase enzyme or a portion thereof, wherein said DNA sequence is detected using a probe comprising an oligonucloetide sequence of at least 16 nucleotides, which hybridizes to the DNA sequence shown in SEQ ID NO: 2, or to the complementary DNA sequence, under at least medium stringency conditions, and preferably at high stringency conditions as defined herein.

Another aspect of the invention relates to an animal feed additive comprising a galactanase as defined in the first or second aspects, or an enzyme preparation as defined in the seventh aspect.

A final aspect of the invention relates to an animal feed composition comprising a galactanase as defined in the first or second aspects, or an enzyme preparation as defined in the seventh.

### DRAWINGS

FIGS. 1 and 2 show dosis-response curves for the B. pumilus NN017642 galactanase and two other galactanases, isolated from B. licheniformis (WO 00/47711) and Humicola insolens (WO 97/32014), in the two different galactanase assays: Reducing sugar assay (FIG. 1) and AZCL assay (FIG. 2).

FIG. 3 shows the pH-activity profile and FIG. 4 shows the pH-stability profile of the B. pumilus, B. licheniformis, and H. insolens galactanases.

In FIG. 5 is shown the temperature activity profile of the galactanases. The galactanase activities were determined at pH 6.5 and at temperatures: 15° C., 25° C., 37° C., 50° C., 65° C., and 75° C. The activities were plotted as relative activities, relative to the activity at the temperature where the galactanase had the highest activity.

Deposited Microorganisms E. coli GAL1019 (NN049518) comprising a Sau3A-fragment ligated into a BamHI digested pZero-2™ (Invitrogen) vector was deposited with DSMZ on the Feb. 4, 2000 under the accession number DSM 13286. The deposit was made by Novo Nordisk A/S and was later assigned to Novozymes A/S. The Sau3A-fragment comprises a polynucleotide encoding the Bacillus pumilus (NN017642) galactanase of the invention.

Definitions

The term “ortholog” (or “species homologue”) denotes a polypeptide or protein obtained from one species that has homology to an analogous polypeptide or protein from a different species.

The term “paralog” denotes a polypeptide or protein obtained from a given species that has homology to a distinct polypeptide or protein from that same species.

The term “expression vector” denotes a DNA molecule, linear or circular, that comprises a segment encoding a polypeptide of interest operably linked to additional segments that provide for its transcription. Such additional segments may include promoter and/or terminator sequences, and may optionally include one or more origins of replication, one or more selectable markers, an enhancer, a polyadenylation signal, and the like. Expression vectors are generally derived from plasmid or viral DNA, or may contain elements of both. The expression vector of the invention may be any expression vector that is conveniently subjected to recombinant DNA procedures, and the choice of vector will often depend on the host cell into which the vector it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The term “recombinant expression” or “recombinantly expressed” used herein in connection with expression of a polypeptide or protein is defined according to the standard definition in the art. Recombinant expression of a protein is generally performed by using an expression vector as described immediately above.

The term “isolated”, when applied to a polynucleotide molecule, denotes that the polynucleotide has been removed from its natural genetic environment and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example Dynan and Tijan, Nature 316:774-78, 1985). The term “an isolated polynucleotide” may alternatively be termed “a cloned polynucleotide”.

When applied to a protein/polypeptide, the term “isolated” indicates that the protein is found in a condition other than its native environment. In a preferred form, the isolated protein is substantially free of other proteins, particularly other homologous proteins (i.e. “homologous impurities” (see below)). It is preferred to provide the protein in a greater than 40% pure form, more preferably greater than 60% pure form. Even more preferably, the protein is provided in a highly purified form, i.e. greater than 80% pure, more preferably greater than 95% pure, and even more preferably greater than 99% pure, as determined by SDS-PAGE.

The term “isolated protein/polypeptide may alternatively be is termed “purified protein/polypeptide”.

The term “homologous impurities” means any impurity (e.g. a polypeptide other than the polypeptide of the invention) which originates from the homologous cell where the polypeptide of the invention is originally obtained from.

The term “obtained from” as used herein in connection with a specific microbial source means that the polynucleotide and/or polypeptide is produced by the specific source, or by a cell in which a gene from the source has been inserted.

The term “operably linked”, when referring to DNA segments, denotes that the segments are arranged so that they function in concert for their intended purposes, e.g. transcription initiates in the promoter and proceeds through the coding segment to the terminator.

The term “polynucleotide” denotes a single- or double-stranded polymer of deoxyribonucleotide or ribonucleotide bases read from the 5′ to the 3′ end. Polynucleotides include RNA and DNA and may be isolated from natural sources, synthesized in vitro, or prepared from a combination of natural and synthetic molecules.

The term “complements of polynucleotide molecules” denotes polynucleotide molecules having a complementary base sequence and reverse orientation as compared to a reference sequence. For example, the sequence 5′ ATGCACGGG 3′ is complementary to 5′ CCCGTGCAT 3′.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (for example, GAU and GAC. triplets both encode Asp).

The term “promoter” denotes a portion of a gene containing DNA sequences that provide for the binding of RNA polymerase and initiation of transcription. Promoter sequences are commonly, but not always, found in the 5′ non-coding regions of genes.

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger peptide is commonly cleaved to remove the secretory peptide during transit through the secretory pathway.

In the present context, the term “galactanase” is defined according to the Enzyme Classification (EC) as having the EC-number 3.2.1.89, the official name arabinogalactan endo-1,4-beta-galactosidase, the alternative names endo-1,4-beta-galactosidase, galactanase and arabinogalactanase, and catalyzing the reaction: endohydrolysis of 1,4-beta-D-galactosidic linkages in galactans or arabinogalactans.

As used herein, the terms DP2, DP3, DP4, etc. are used to refer to oligosaccharides consisting of the indicated number of monosaccharide groups, e.g. DP4 refers to galactotetrabiose. In monogastric animals the non-starch polysaccharides are not degraded in the small intestine by the digestive enzymes, and hence do not offer their full energy potential to the animal. Feed enhancing enzymes are enzymes that by improving feed digestibility are able to increase the efficiency of the feed utilization. Feed enhancing enzymes function by enhancing the digestibility of feed components. This enhancement may e.g. be brought about by degradation of polysaccharides. In e.g. soybean meal, galactans constitute a significant part of the pectinous non-starch polysaccharides.

Complete degradation of galactans can be achieved e.g. by a combination of galactanase degradation, to form oligosaccharides such as DP4, and beta-galactosidase mediated degradation which results in free galactose that can be absorbed from the small is intestine. Alternatively, cell wall non-starch polysaccharides may act to encapsulate or bind other nutrients, and therefore partial degradation of these polysaccharides can increase the availability of nutrients.

The term “animal” includes all animals, including human beings. Examples of animals are mono-gastric animals, e.g. pigs (including, but not limited to, piglets, growing pigs, and sows); poultry such as turkeys and chicken (including but not limited to broiler chicks, layers); young calves; and fish (including but not limited to salmon).

The term “feed” or “feed composition” means any compound, preparation, mixture, or composition suitable for, or intended for intake by an animal. In a particular embodiment, one or more galactan sources are included in the feed. These may derive from e.g. soybeans, lupins or potatoes.

The term “improving the nutritional value of an animal feed” means improving the solubilisation and/or degradation of the galactans, thereby leading to increased availability of nutrients. The nutritional value of the feed is therefore increased, and the growth rate and/or feed conversion ratio (i.e. the weight of ingested feed relative to weight gain) of the animal is improved.

The term “modifying an animal feed” means altering the properties of the feed such as viscosity or nutritional value.

### DETAILED DESCRIPTION OF THE INVENTION

The inventors isolated a novel galactanase with improved properties from a species of Bacillus.

A preferred embodiment of the invention is an isolated polypeptide having galactanase activity according to the first or second aspects of the invention, wherein the polypeptide is isolated from a Bacillus cell, preferably a Bacillus pumilus cell.

Further a preferred embodiment of the invention relates to the isolated polypeptide according to the first or second aspects, which has an activity optimum at a pH-value between 5.5 and 7.5, preferably between 6 and 7, as measured herein.

Another preferred embodiment of the invention relates to the isolated polypeptide according to the first or second aspects, which has an optimum activity at a temperature between 30° C. and 70° C., preferably between 35° C. and 65° C., more preferably between 37° C. and 60° C., even more preferably between 42° C. and 58° C., and most preferably between 45° C. and 55° C. as measured herein.

Yet another preferred embodiment of the invention relates to the isolated polypeptide according to the second aspect, which maintains at least 30%, preferably 40%, more preferably 45%, and most preferably 50% residual galactanase activity after two hours at a temperature of 37° C. at pH 4.0, measured as defined herein.

Another preferred embodiment of the invention relates to the isolated polypeptide according to the second aspect, which maintains at least 30%, preferably 40%, more preferably 50%, and most preferably 60% residual galactanase activity after two hours at a temperature of 37° C. and at pH 4.5, measured as defined herein.

Yet another preferred embodiment of the invention relates to the isolated polypeptide according to the second aspect, which maintains at least 30%, preferably 40%, more preferably 50%, and most preferably 60% residual galactanase activity after two hours at a temperature of 37° C. and at pH 3.5, measured as defined herein.

The activity of a galactanase of the invention was characterized as described below, and it was found to release mainly DP4 oligogalactan residues when degrading galactan; further it was characterized with respect to pH and temperature.

Thus a preferred embodiment of the invention relates to the isolated polypeptide according to the first aspect, which mainly releases DP4 oligogalactans when degrading galactan.

Further a preferred embodiment of the invention relates to an isolated polynucleotide molecule encoding a polypeptide having galactanase activity according to the third aspect of the invention, wherein the polynucleotide is DNA.

One more preferred embodiment of the invention relates to an enzyme preparation comprising an isolated polypeptide of the seventh aspect, which further comprises one or more enzymes selected from the group consisting of proteases, cellulases (endoglucanases), β-glucanases, hemicellulases, lipases, peroxidases, laccases, α-amylases, glucoamylases, cutinases, pectinases, reductases, oxidases, phenoloxidases, ligninases, pullulanases, arabinases, mannanases, xyloglucanases, xylanases, pectin acetyl esterases, polygalacturonases, rhamnogalacturonases, pectin lyases, pectate lyases, pectin methylesterases, cellobiohydrolases, transglutaminases, and phytases; or mixtures thereof.

A preferred embodiment of the invention relates to an enzyme preparation comprising an isolated polypeptide of the seventh aspect, which further comprises one or more enzymes selected from the group consisting of proteases, carbohydrases, lipases, reductases, oxidases, transglutaminases, and phytases; or mixtures thereof.

In a preferred embodiment, one or more of the enzymes in the preparation is produced by using recombinant techniques, i.e. the enzyme is a mono-component enzyme which is mixed with at least one other enzyme to form an enzyme preparation with the desired enzyme blend.

Still another preferred embodiment of the invention relates to a method for modifying and/or improving the nutritional value of an animal feed according to the eigth aspect wherein the galactanase added to the animal feed is an analogue of the enzyme defined in a), said analogue being at least 70% identical with said enzyme in a), preferably at least 80% identical, more preferably at least 90% identical, and most preferably at least 95% identical, and preferably the animal feed comprises plant material, more preferably the plant material comprises soybean (Glycine max), even more preferably the galactanase added to the animal feed is of bacterial origin, and most preferably the galactanase is derived from a strain of Bacillus.

In a preferred embodiment the invention relates to a method for modifying and/or improving the nutritional value of an animal feed according to the ninth aspect of the invention, wherein the animal feed comprises plant material, preferably the plant material comprises soybean (Glycine max), and more preferably wherein to the animal feed further is added one or more enzymes selected from the group consisting of proteases, cellulases (endoglucanases), β-glucanases, hemicellulases, lipases, peroxidases, laccases, α-amylases, glucoamylases, cutinases, pectinases, reductases, oxidases, phenoloxidases, ligninases, pullulanases, arabinases, mannanases, xyloglucanases, xylanases, pectin acetyl esterases, polygalacturonases, rhamnogalacturonases, pectin lyases, pectate lyases, pectin methylesterases, cellobiohydrolases, transglutaminases, and phytases; or mixtures thereof.

In a final preferred embodiment the invention relates to a method for modifying and/or improving the nutritional value of an animal feed according to the ninth aspect of the invention, wherein the animal feed comprises plant material, preferably the plant material comprises soybean (Glycine max), and more preferably wherein to the animal feed further is added one or more enzymes selected from the group consisting of proteases, carbohydrases, lipases, reductases, oxidases, transglutaminases, and phytases; or mixtures thereof.

Using a Sequence of the Invention to Obtain Other Related Sequences

The disclosed sequence information herein relating to polynucleotide sequences encoding galactanases of the invention can be used as a tool to identify other homologous galactanase sequences. For instance, PCR (polymerase chain reaction) can be used to amplify sequences encoding other homologous galactanases from a variety of other microbial sources of in particular, but not limited to, different Bacillus species. As primers in the PCR reactions, DNA oligonucleotides consisting of, e.g., 16 or more bases of the above listed primers can be used, either in combination with another primer related to the sequences of the invention, or in combination with any other primer useful for amplifying a PCR fragment.

Assay for Activity

A polypeptide of the invention having galactanase activity may be assayed for galactanase activity according to standard assay procedures known in the art, such as by applying a solution to be tested to 4 mm diameter holes punched out in agar plates containing 0.2% AZCL galactan (Megazyme, Ireland).

Polynucleotides

Species homologues of a polypeptide of the invention having galactanase activity can be cloned using information and compositions provided by the present invention in combination with conventional cloning techniques. For example, DNA can be cloned using chromosomal DNA obtained from a cell type that expresses the protein. Suitable sources of DNA can be identified by probing Northern blots with probes designed from the sequences disclosed herein. A library is then prepared from chromosomal DNA of a positive cell line. DNA encoding an polypeptide of the invention having galactanase activity can then be isolated by a variety of methods, such as by probing with a complete or partial DNA or with one or more sets of degenerate probes based on the disclosed sequences. DNA can also be cloned using the polymerase chain reaction, or PCR (Mullis, U.S. Pat. No. 4,683,202), using primers designed from the sequences disclosed herein. Within an additional method, the DNA library can be used to transform or transfect host cells, and expression of the DNA of interest can be detected with an antibody (monoclonal or polyclonal) raised against the galactanase cloned from B. pumilus, expressed and purified as described in examples, or by an activity test relating to a polypeptide having galactanase activity.

In particular polynucleotides of the invention will hybridize to a double-stranded DNA probe comprising the sequence shown in: positions 1-1209 in SEQ ID NO: 2 under at least medium stringency conditions, but preferably at high stringency conditions as described in detail below. Suitable experimental conditions for determining hybridization at medium, or high stringency between a nucleotide probe and a homologous DNA or RNA sequence involves presoaking of the filter containing the DNA fragments or RNA to hybridize in 5×SSC. (Sodium chloride/Sodium citrate, Sambrook et al. 1989) for 10 min, and prehybridization of the filter in a solution of 5×SSC., 5×Denhardt's solution (Sambrook et al. 1989), 0.5% SDS and 100 μg/ml of denatured sonicated salmon sperm DNA (Sambrook et al. 1989), followed by hybridization in the same solution containing a concentration of 10 ng/ml of a random-primed (Feinberg, A. P. and Vogelstein, B. (1983) Anal. Biochem. 132:6-13), 32P-dCTP-labeled (specific activity>1×109 cpm/μg) probe for 12 hours at ca. 45° C. The filter is then washed twice for 30 minutes in 2×SSC., 0.5% SDS at least 60° C. (medium stringency), still more preferably at least 65° C. (medium/high stringency), even more preferably at least 70° C. (high stringency), and even more preferably at least 75° C. (very high stringency).

Molecules to which the oligonucleotide probe hybridizes under these conditions are detected using a x-ray film.

As previously noted, the isolated polynucleotides of the present invention include DNA and RNA. Methods for isolating DNA and RNA are well known in the art. DNA and RNA encoding genes of interest can be cloned in Gene Banks or DNA libraries by means of methods known in the art.

Polynucleotides encoding polypeptides having galactanase activity of the invention are then identified and isolated by, for example, hybridization or PCR.

The present invention further relates to polypeptides and polynucleotides from different bacterial strains (orthologs or paralogs). Of particular interest are galactanase polypeptides from Gram-positive strains, including species of Bacillus such as Bacillus subtilis, Bacillus lentus, Bacillus brevis, Bacillus stearothermophilus, Bacillus alkalophilus, Bacillus amyloliquefaciens, Bacillus coagulans, Bacillus circulans, Bacillus lautus, Bacillus thuringiensis, Bacillus licheniformis, or in particular Bacillus pumilus.

Polypeptides

Suitable galactanase polypeptides for purposes of the present invention are those that are substantially homologous to the polypeptides identified above and their species homologues (paralogs or orthologs). The term “substantially homologous” is used herein to denote polypeptides having at least 70%, preferably at least 80%, more preferably at least 85%, and even more preferably at least 90%, sequence identity to the sequences shown herein or their orthologs or paralogs. Such polypeptides will more preferably be at least 95% identical, and most preferably 98% or more identical to such sequences or their orthologs or paralogs. Percent sequence identity may be determined by conventional methods, e.g. the “align” program discussed herein.

The sequence of amino acids no. 1-402 of SEQ ID NO: 3 is a mature galactanase sequence. The present invention also provides galactanase polypeptides that are substantially homologous to the polypeptides of SEQ ID NO: 3 and their species homologs (paralogs or orthologs).

Substantially homologous proteins and polypeptides are characterized as having one or more amino acid substitutions, deletions or additions. These changes are preferably of a minor nature, that is conservative amino acid substitutions (see Table 2) and other substitutions that do not significantly affect the folding or activity of the protein or polypeptide; small deletions, typically of from one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A (Nilsson et al., EMBO J. 4:1075, 1985; Nilsson et al., Methods Enzymol. 198:3, 1991. See in general Ford et al., Protein Expression and Purification 2: 95-107, 1991, which is incorporated herein by reference. DNAs encoding affinity tags are available from commercial suppliers (e.g. Amersham Pharmacia, Piscataway, N.J., USA; New England Biolabs, Beverly, Mass, USA).

However, even though the changes described above preferably are of a minor nature, such changes may also be of a more substantial nature such as fusion of larger polypeptides of up to 300 amino acids or more as amino- or carboxyl-terminal extensions to a galactanase polypeptide of the invention.

TABLE 2
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and a-methyl serine) may be substituted for amino acid residues of a polypeptide according to the invention. A limited number of non-conservative amino acids, amino acids that are not encoded by the genetic code, and/or unnatural amino acids may be substituted for amino acid residues. “Unnatural amino acids” have been modified after protein synthesis, and/or have a chemical structure in their side chain(s) different from that of the standard amino acids. Unnatural amino acids can be chemically synthesized, or preferably, are commercially available, and include pipecolic acid, thiazolidine carboxylic acid, dehydroproline, 3- and 4-methylproline, and 3,3-dimethylproline.

Essential amino acids in the galactanase polypeptides of the present invention can be identified according to procedures known in the art, such as site-directed mutagenesis or alanine-scanning mutagenesis (Cunningham and Wells, Science 244: 1081-1085, 1989). In the latter technique, single alanine mutations are introduced at every residue in the molecule, and the resultant mutant molecules are tested for biological activity (i.e galactanase activity) to identify amino acid residues that are critical to the activity of the molecule. See also, Hilton et al., J. Biol. Chem. 271:4699-4708, 1996. The active site of the enzyme or other biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-312, 1992; Smith et al., J. Mol. Biol. 224:899-904, 1992; Wlodaver et al., FEBS Lett. 309:59-64, 1992. The identities of essential amino acids can also be inferred from analysis of homologies with polypeptides which are related to a polypeptide according to the invention.

Multiple amino acid substitutions can be made and tested using known methods of mutagenesis, recombination and/or shuffling followed by a relevant screening procedure, such as those disclosed by Reidhaar-Olson and Sauer (Science 241:53-57, 1988), Bowie and Sauer (Proc. Natl. Acad. Sci. USA 86:2152-2156, 1989), WO 95/17413, or WO 95/22625. Briefly, these authors disclose methods for simultaneously randomizing two or more positions in a polypeptide, or recombination/shuffling of different mutations (WO 95/17413, WO 95/22625), followed by selecting for a functional polypeptide, and then sequencing the mutated polypeptides to determine the spectrum of allowable substitutions at each position. Other methods that can be used include phage display (e.g., Lowman et al., Biochem. 30:10832-10837, 1991; Ladner et al., U.S. Pat. No. 5,223,409; Huse, WO 92/06204) and region-directed mutagenesis (Derbyshire et al., Gene 46:145, 1986; Ner et al., DNA 7:127, 1988).

Mutagenesis/shuffling methods as disclosed above can be combined with high-throughput, automated screening methods to detect activity of cloned, mutagenized polypeptides in host cells. Mutagenized DNA molecules that encode active polypeptides can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can identify and/or prepare a variety of polypeptides that are substantially homologous to those disclosed herein and retain the galactanase activity of the wild-type protein.

Protein Production

The polypeptides of the present invention, including full-length proteins, fragments thereof and fusion proteins, can be produced in genetically engineered host cells according to conventional techniques. Suitable host cells are those cell types that can be transformed or transfected with exogenous DNA and grown in culture, and include bacteria, fungal cells, and cultured higher eukaryotic cells. Bacterial cells, particularly cultured cells of gram-positive organisms, are preferred. Gram-positive cells from the genus Bacillus are especially preferred, such as B. subtilis, B. lentus, B. brevis, B. stearothermophilus, B. alkalophilus, B. amyloliquefaciens, B. coagulans, B. circulans, B. lautus, B. thuringiensis, B. agaradhaerens, B. pumilus and B. licheniformis.

Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, NY, 1989; Ausubel et al. (eds.), Current Protocols in Molecular Biology, John Wiley and Sons, Inc., NY, 1987; and Bacillus subtilis and Other Gram-Positive Bacteria, Sonensheim et al., 1993, American Society for Microbiology, Washington D.C.; which are incorporated herein by reference.

In general, a DNA sequence encoding a galactanase of the present invention is operably linked to other genetic elements required for its expression, generally including a transcription promoter and terminator within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design for those of ordinary skill in the art. Many such elements are described in the literature and are available through commercial suppliers.

To direct a polypeptide into the secretory pathway of a host cell, a secretory signal sequence (also known as a leader sequence, prepro sequence or pre sequence) is provided in the expression vector. The secretory signal sequence may be that of the polypeptide or may be derived from another secreted protein or synthesized de novo. Numerous suitable secretory signal sequences are known in the art and reference is made to the following for further description of suitable secretory signal sequences, especially for secretion in a Bacillus host cell: Sonensheim et al., 1993; and Cutting, S. M.(eds.) “Molecular Biological Methods for Bacillus”, John Wiley and Sons, 1990. The secretory signal sequence is joined to the DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum as required. The growth medium will generally select for cells containing the exogenously added DNA by, for example, drug selection or deficiency in an essential nutrient which is complemented by the selectable marker carried on the expression vector or co-transfected into the host cell.

Protein Isolation

When the expressed recombinant polypeptide is secreted, the polypeptide may be purified from the growth media. Preferably, the expression host cells are removed from the media before purification of the polypeptide (e.g. by centrifugation).

When the expressed recombinant polypeptide is not secreted from the host cell, the host cell is preferably disrupted and the polypeptide released into an aqueous “extract” which is the first stage of such purification techniques. Preferably, the expression host cells are removed from the media before the cell disruption, e.g. by centrifugation.

The cell disruption may be performed by conventional techniques such as by lysozyme digestion or by forcing the cells through high pressure. See e.g. Robert K. Scobes, Protein Purification, Second edition, Springer-Verlag, for further description of such cell disruption techniques.

Regardless of whether the expressed recombinant polypeptides (or chimeric polypeptides) are secreted or not, they can be purified using fractionation and/or conventional purification on chromatographic media.

Fractionation can be achieved by precipitation of the polypeptides with e.g. ammonium sulfate, organic solvents, PEG (polyethylene glycol) or PEI (polyethyleneimine) or by a selective denaturaturation of impurities, e.g. by adjusting pH and/or temperature.

Purification by liquid chromatography may include hydroxyapatite chromatography, size exclusion chromatography, ion-exchange chromatography, hydrophobic interaction chomatography and/or affinity chromatography. Chromatography media consists of a hydrophilic insoluble matrix (or support) to which ligands are attached (except size exclusion media, where no ligands are attached). Suitable matrixes include: agarose, cellulose, dextran, polyacrylamide, polystyrene, methacrylate, controlled pore glass, silica based resins, and the like. Often the matrixes are crosslinked to reduce their resistance to flow and in some cases the surface of the matrices is derivatized or coated with a hydrophilic polymer to avoid unspecific binding of biomolecules to the matrixes. Anion exchange media are derivatized with cationic ligands: PEI, DEAE, QAE or Q, such as DEAE sepharose FF (Amersham Pharmacia Biotech), cation exchange media with anionic ligands: CM, SP or S, such as SP sepharose FF (Amersham Pharmacia Biotech), and hydrophobic interaction media with hydrophobic ligands: phenyl, butyl, isopropyl or octyl groups, such as Toyopearl butyl 650 (TosoHaas). The matrices may also be modified with reactive groups that allow attachment of proteins (or other types of ligands) thought their amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activiation, hydrazide activation, and carboxyl and amino derivatives for carbodiimide coupling chemistries. Selection of a particular method for attachment is a matter of routine design and is determined in part by the properties of the chosen support and in part by the properties of the immobilized protein (or ligand). See, for example, Affinity Chromatography: Principles & Methods, Pharmacia LKB Biotechnology, Uppsala, Sweden, 1988.

Chromatographic media are well known and widely used in the art, and are available from a range of commercial suppliers.

Polypeptides of the invention or fragments thereof may also be prepared through chemical synthesis. Polypeptides of the invention may be monomers or multimers; glycosylated or non-glycosylated; pegylated or non-pegylated; and may or may not include an initial methionine amino acid residue.

In the present context, the term “enzyme preparation” is intended to mean either be a conventional enzymatic fermentation product, possibly isolated and purified, from a single species of a microorganism, such preparation usually comprising a number of different enzymatic activities; or a mixture of monocomponent enzymes, preferably enzymes derived from bacterial or fungal species by using conventional recombinant techniques, which enzymes have been fermented and possibly isolated and purified separately and which may originate from different species, preferably fungal or bacterial species; or the fermentation product of a microorganism which acts as a host cell for expression of a recombinant galactanase, but which microorganism simultaneously produces other enzymes, e.g. galactanases, proteases, or cellulases, being naturally occurring fermentation products of the microorganism, i.e. the enzyme complex conventionally produced by the corresponding naturally occurring microorganism.

The enzymes used according to the invention may further be produced by culturing a wild-type microorganism, or a microorganism altered by mutation, capable of producing the galactanase under conditions permitting the production of the enzyme, and recovering the enzyme from the culture. Culturing may be carried out using conventional fermentation techniques, e.g. culturing in shake flasks or fermentors with agitation to ensure sufficient aeration on a growth medium inducing production of the galactanase enzyme. The growth medium may contain a conventional N-source such as peptone, yeast extract or casamino acids, a reduced amount of a conventional C-source such as dextrose or sucrose, and an inducer such as galactan or plant substrates such as soybean or lupin. The recovery may be carried out using conventional techniques, e.g. separation of bio-mass and supernatant by centrifugation or filtration, recovery of the supernatant or disruption of cells if the enzyme of interest is intracellular, optionally followed by further purification e.g. as described in EP 0 406 314 or by crystallization as described in WO 97/15660.

Animal Feed Additive

As indicated above, the galactanases of the present invention are particularly suited for use as an animal feed additive for modification of the animal feed. The effect of the galactanases can be exerted either in vitro (by pretreatment of components of the feed) or in vivo. The galactanases are particularly suited for addition to animal feed compositions containing high amounts of arabinogalactans or galactans, e.g. feed containing plant material from soy bean, rape seed, lupin etc. When added to the feed, the galactanase significantly improves the in vivo break-down of plant cell wall material, whereby a better utilization of the plant nutrients by the animal is achieved. Thereby, the growth rate and/or feed conversion ratio (i.e. the weight of ingested feed relative to weight gain) of the animal is improved. For example, the indigestible galactan is degraded by galactanase, e.g. in combination with β-galactosidase, to galactose or galactooligomers which are digestible by the animal and thus contribute to the available energy of the feed. Also, by degrading galactan, the galactanase may improve the digestibility and uptake of non-galactan feed constituents such as protein, fat and minerals

It is anticipated that the method of using a galactanase of the invention as an animal feed additive may be improved by further adding one or more enzymes selected from the group consisting of proteases, carbohydrases, lipases, reductases, oxidases, transglutaminases, and phytases; or mixtures thereof. Additional uses for the galactanases of the invention include the following.

Degradation or Modification of Plant Material

The galactanases according to the invention may be used as an agent for degradation or modification of plant cell walls or any galactan-containing material originating from plant cells walls due to the high plant cell wall degrading activity of the enzymes.

The degradation of galactan by galactanases is facilitated by full or partial removal of the sidebranches. Arabinose sidegroups can be removed by a mild acid treatment or by alpha-arabinosidases. The oligomers with are released by the galactanase or by a combination of galactanases and sidebranch-hydrolysing enzymes as mentioned above can be further degraded to free galactose by beta-galactosidases.

The galactanase of the present invention can be used without other pectinolytic or hemicellulytic enzymes or with limited activity of other pectinolytic or hemicellulytic enzymes to degrade galactans for production of oligosaccharides. The oligosaccharides may be used as bulking agents, like arabinogalactan oligosaccharides released from soy cell wall material, or of more or less purified arabinogalactans from plant material.

The galactanases of the present invention can be used in combination with other pectinolytic or hemicellulytic enzymes to degrade galactans to galactose and other monosaccharides.

The galactanase of the present invention may be used alone or together with other enzymes like glucanases, pectinases and/or hemicellulases to improve the extraction of oil from oil-rich plant material, like soy-bean oil from soy-beans, olive-oil from olives or rapeseed-oil from rape-seed or sunflower oil from sunflower.

The galactanases of the present invention may be used for separation of components of plant cell materials. Of particular interest is the separation of sugar or starch rich plant material into components of considerable commercial interest (like sucrose from sugar beet or starch from potato) and components of low interest (like pulp or hull fractions). Also, of particular interest is the separation of protein-rich or oil-rich crops into valuable protein and oil and invaluable hull fractions, The separation process may be performed by use of methods known in the art.

The galactanases of the invention may also be used in the preparation of fruit or vegetable juice in order to increase yield, and in the enzymatic hydrolysis of various plant cell wall-derived materials or waste materials, e.g. from wine or juice production, or agricultural residues such as vegetable hulls, bean hulls, sugar beet pulp, olive pulp, potato pulp, and the like.

The plant material may be degraded in order to improve different kinds of processing, facilitate purification or extraction of other components than the galactans like purification of pectins from citrus, improve the feed value, decrease the water binding capacity, improve the degradability in waste water plants, improve the conversion of plant material to ensilage, etc.

By means of an enzyme preparation of the invention it is possible to regulate the consistency and appearance of processed fruit or vegetables. The consistency and appearance has been shown to be a product of the actual combination of enzymes used for processing, i.e. the specificity of the enzymes with which the galactanase of the invention is combined. Examples include the production of clear juice e.g. from apples, pears or berries; cloud stable juice e.g. from apples, pears, berries, citrus or tomatoes; and purees e.g. from carrots and tomatoes.

The galactanases of the invention may be used in modifying the viscosity of plant cell wall derived material. For instance, the galactanase may be used to reduce the viscosity of feed which contain galactan and to promote processing of viscous galactan containing material. The viscosity reduction may be obtained by treating the galactan containing plant material with an enzyme preparation of the invention under suitable conditions for full or partial degradation of the galactan containing material.

Wine and Juice Processing

The enzyme or enzyme preparation of the invention may be used for de-pectinization and viscosity reduction in vegetable or fruit juice, especially in apple or pear juice. This may be accomplished by treating the fruit or vegetable juice with an enzyme preparation of the invention in an amount effective for degrading pectin-containing material contained in the fruit or vegetable juice.

The enzyme or enzyme preparation may be used in the treatment of mash from fruits and vegetables in order to improve the extractability or degradability of the mash. For instance, the enzyme preparation may be used in the treatment of mash from apples and pears for juice production, and in the mash treatment of grapes for wine production.

Use in the Detergent Industry

In further aspects, the present invention relates to a detergent composition comprising the galactanases or galactanase preparation of the invention, and to a process for machine treatment of fabrics comprising treating fabric during a washing cycle of a machine washing process with a washing solution containing the galactanase or galactanase preparation of the invention.

Typically, the detergent composition of the invention comprises conventional ingredients such as surfactants (anionic, nonionic, zwitterionic, amphoteric), builders, and other ingredients, e.g. as described in WO 97/01629 which is hereby incorporated by reference.

Use in the Textile and Cellulosic Fiber Processing Industries

The galactanases of the present invention can be used in combination with other enzymes such as carbohydrate degrading enzymes (for instance arabinanase, xyloglucanase, pectinase) for preparation of textile fibres or other cellulosic materials fibers or for cleaning of fibers in combination with detergents. Cotton fibers consist of a primary cell wall layer containing pectin and a secondary layer containing mainly cellulose. Under cotton preparation or cotton refining part of the primary cell wall will be removed. The present invention relates to either help during cotton refining by removal of the primary cell wall or during cleaning of the cotton to remove residual pectic substances and prevent graying of the textile. For more information on textile and treatment of textile with enzymes of the invention WO 99/27083 is incorporated herein by reference.

The invention will be further illustrated by the following non-limiting examples.

### EXAMPLES

Materials and Methods

BCA Protein Assay

This assay is identical to PIERCE cat. No. 23225: BCA protein assay kit. The BCA working solution was made by mixing 50 parts of reagent A with 1 part reagent B. 20 μl sample was mixed with 200 μl BCA working solution. After 30 minutes at 37° C., the sample was cooled to room temperature and OD₄₉₀ was read as a measure of the protein concentration in the sample. Dilutions of BSA were included in the assay as a standard.

Reducing Sugar Assay

15 mg/ml arabinofuranosidase treated lupin galactan (from Megazyme with a 91:2:1.7:0.3:5 ratio between Gal:Ara:Rha:Xyl:GalUA) was dissolved in 5 mM CH₃COOH, 50 mM KH₂PO₄, 5 mM H₃BO₃, 1 mM CaCl₂, 0.01% Triton X-100, pH 6.5 (assay buffer) and adjusted to pH 6.5 with NaOH (galactan substrate). 50 μl galactan substrate and 25 μl galactanase sample (diluted in assay buffer) were mixed in an Eppendorf tube placed on ice. The assay was initiated by transferring the Eppendorf tube to an Eppendorf thermomixer, which was set to the assay temperature (37° C.). The tube was incubated for 15 minutes on the Eppendorf thermomixer at a moderate shaking rate. The incubation was stopped by transferring the tube back to the ice bath and adding 500 μl ice cold CNC-reagent. The stopped incubation was mixed by pipetting. The temperature was brought to 25° C. by a 5 minutes incubation on an Eppendorf thermomixer, which was set to 25° C. (moderate mixing in the dark). Finally, 200 μl mixture was transferred to a microtiter plate and after 20 to 30 minutes at room temperature in the dark, OD₄₅₀ was read as a measure of galactanase activity.

Buffer blinds and galactose dilutions were included in the assay as standards (instead of enzyme).

The CNC-reagent was made by mixing in the following order: 1.8 ml deionised water, 200 μl 40 mM CuSO₄, 6.0 ml 2.75 mM Neocuproin-HCl, and 2.67 ml 2.0 M Na₂CO₃. The CNC-reagent was made just before use.

The galactase activities determined by the above reducing sugar assay are indicated as galu/ml. 1 galu is defined as the amount of enzyme, that creates reducing ends equivalent to 1 μmol galactose per minute.

AZCL Assay

4 mg/ml AZCL lupin galactan (from Megazyme) was homogenously suspended in deionised water by stirring. 500 μl AZCL galactan suspension and 500 μl assay buffer (50 mM CH₃COOH, 50 mM KH₂PO₄, 50 mM H₃BO₃, 1 mM CaCl₂, 0.01% Triton X-100, adjusted to the relevant pH-values with HCl or NaOH) were mixed in an Eppendorf tube placed on ice. 20 μl galactanase sample (diluted in assay buffer) was added. The assay was initiated by transferring the Eppendorf tube to an Eppendorf thermomixer, which was set to the assay temperature. The tube was incubated for 15 minutes on the Eppendorf thermomixer at its highest shaking rate. The incubation was stopped by transferring the tube back to the ice bath. Then the tube was centrifuged in an icecold centrifuge for a few minutes and 200 μl supernatant was transferred to a microtiter plate. OD₆₅₀ was read as a measure of galactanase activity. A buffer blind was included in the assay (instead of enzyme).

### EXAMPLE 1

Isolation and Growth of of Galactanase Producing Strain

A marine sediment sample collected close to the Danish island RØmØ was plated on marine agar (Difco 2216) and incubated at 37° for 2 days. Colonies resembling members of the genus Bacillus were collected and characterized. One isolate NN017642 identified as Bacillus pumilus by partial 16Sr-RNA sequence analysis showed galactanase activity on the substrate AZCL-galactan (Megazyme) by forming blue haloes around the colonies.

The strain was grown overnight at 30° C. in tryptone yeast extract medium with 3% sodium chloride, centrifuged at 4000 rpm for 15 minutes, the supernatant discarded and the cell pellet used for DNA isolation as described below.

### EXAMPLE 2

Preparation of Gene Library

The cells were resuspended in 7.5 ml of buffer with the following composition: 10 mM NaCl, 20 mM Tris/HCl, 1 mM EDTA, 0.5% SDS and 100 microgram/ml Proteinase K and incubated overnight at 50° C.

7.5 ml Phenol:Chloroform:isoamylalkohol (25:24:1) was added, the sample mixed and centrifuged at 4000 rpm for 30 minutes. The upper phase after centrifugation was transferred to a fresh tube and the Phenol:Chloroform:isoamylalkohol treatment repeated. The resulting upper phase was mixed with 7.5 ml chloroform and centrifuged at 4000 rpm for 10 minutes. The upper phase was separated, mixed with 1/10 volume 3 M sodium acetate and 2 volumes 96% ethanol and left on ice for precipitation of DNA. The DNA was collected with a glass hook, washed with 70% ethanol and resuspended in 1 ml TE buffer, pH 8.

The DNA was partially digested with the restriction enzyme Sau3A and the DNA was size-fractionated on a 1% agarose gel (Maniatis et al.). The agarose containing the DNA corresponding to 3kb and upwards in size was cut from the agarose gel and the DNA was concentrated by further electrophoresis into a 1.2% agarose gel. The DNA was isolated from the agarose piece using the GFX kit (Pharmacia).

The Sau3A digested DNA was ligated into a fresh BamHI digested pZero-2 (Invitrogen). The ligation mixture was transformed into DH10B E. coli cells by electroporation and frozen in aliqouts corresponding to 300 kanamycin resistant colonies. The frozen aliquots constitute the library.

### EXAMPLE 3

Isolation of Galactanase Positive Clones

An aliquot of the library was diluted to 3 cells/ml in LB medium with 25 microgram/ml kanamycin and distributed into 100 96 well microtiterplates with 300 μl pr well. The plates were incubated with shaking at 37° C. for 40 hours, an 50 μl aliquot was transferred to fresh assay plates and assayed for galactanase activity by addition of 150 μl assay mix with the following composition: 0,075% AZCL potato Galactan (Megazyme)in 0.1 M sodium phosphate buffer pH7,0 followed by overnight incubation at 37° C.

Positive clones give blue color. Several positive clones were obtained, one such clone denoted E. coli GAL1019 (NN049518) was purified and used in the determination of the galactanase gene sequence, the clone NN049518 was deposited with DSMZ on Feb. 4, 2000 under the deposition number DSM 13286.

### EXAMPLE 4

Determination of the Galactanase Gene Sequence

The sequence of the Bacillus pumilus NN017642 galactanase gene in the E. coli clone GAL1019 (DSM 13286) was determined by the GPS™-1 Genome priming system (New England Biolabs, cat. 7100) kit according to manufacturers instruction manual using an ABI Automated Sequencer for the individual sequence determinations.

### EXAMPLE 5

Analysis of the Galactanase Sequence

Comparison of different galactanase amino acid and DNA sequences was done by using the program “align” to calculate individual amino acid and DNA homologies for a number of different galactanase enzymes of both bacterial and fungal origin. The enzymes were isolated from the following microorganisms: Aspergillus aculeatus, Bacillus agaradhaerens, Bacillus circulans, Bacillus licheniformis, Bacillus subtilis, Bacillus pumilus NN017642, Humicola insolens, Meripilus giganteus, Myceliophthora thermophila and Pseudomonas fluorescens. All sequences are known in the public domain as described in Table 1, and the Bacillus pumilus NN017642 (DSM 13286) galactanase sequence is presented herein.

“align” is a full Smith-Waterman alignment, useful for both protein and DNA alignments. The default scoring matrices BLOSUM50 and the identity matrix are used for protein and DNA alignments, respectively. The penalty for the first residue in a gap is −12 for proteins and −16 for DNA. While the penalty for additional residues in a gap is −2 for proteins and −4 for DNA. Align is from the fasta package version v20u6 (William R. Pearson, Department of Biochemistry, Box 440, Jordan Hall University of Virginia, Charlottesville, Va., USA).

Multiple alignments of protein sequences were done using “clustalw” (Thompson, J. D., Higgins, D. G. and Gibson, T. J. (1994) CLUSTAL W: improving the sensitivity of progressive multiple sequence alignment through sequence weighting, positions-specific gap penalties and weight matrix choice. Nucleic Acids Research, 22:4673-4680).

Multiple alignment of DNA sequences were done using the protein alignment as a template, replacing the amino acids with the corresponding codon from the DNA sequence.

The homologies obtained are shown in Table 3 below.

TABLE 3
DNA homologies are read from the top and amino acid homologies are read from the left.

| Protein\DNA | A_acu | B_aga | B_cir | B_lic | B_sub | H_ins | M_gig | M_the | P_flu | B_pum |
|---|---|---|---|---|---|---|---|---|---|---|
| A_aculeatus | 100.0 | 48.1 | 48.7 | 47.0 | 45.6 | 61.5 | 56.1 | 60.4 | 47.2 | 46.6 |
| B_agaradhaerens | 29.2 | 100.0 | 51.5 | 47.8 | 45.0 | 44.5 | 45.2 | 46.2 | 46.1 | 48.7 |
| B_circulans | 30.7 | 43.6 | 100.0 | 49.9 | 47.5 | 47.0 | 48.7 | 48.2 | 51.2 | 51.5 |
| B_licheniformis | 26.5 | 31.3 | 31.5 | 100.0 | 69.4 | 47.7 | 48.3 | 46.7 | 49.0 | 50.5 |
| B_subtilis | 26.5 | 29.0 | 29.9 | 72.5 | 100.0 | 44.0 | 45.7 | 45.9 | 48.1 | 51.2 |
| H_insolens | 55.3 | 29.5 | 30.1 | 29.9 | 26.3 | 100.0 | 57.5 | 74.5 | 49.3 | 47.2 |
| M_giganteus | 45.2 | 26.3 | 30.3 | 26.9 | 25.8 | 42.8 | 100.0 | 57.4 | 49.2 | 47.1 |
| M_thermophila | 54.5 | 27.9 | 31.1 | 29.7 | 27.9 | 75.3 | 46.2 | 100.0 | 49.1 | 48.3 |
| P_fluorescens | 28.5 | 29.2 | 33.8 | 30.9 | 31.5 | 28.3 | 30.1 | 28.4 | 100.0 | 51.7 |
| B_pumilus | 26.4 | 40.0 | 39.0 | 38.3 | 37.3 | 30.8 | 29.6 | 31.7 | 36.6 | 100.0 |

### EXAMPLE 6

Purification of Bacillus pumilus Galactanase

Bacillus pumilus NN017642 was inoculated from tryptone yeast agar slants into 10 shake flasks each containing 100 ml BPX medium with the following composition: potato starch 100 g/l, barley flour 50 g/l, sodium caseinate 10 g/l, soy meal 20 g/l, disodium phosphate 9 g/l and fermented at 30 degree C. for 3 days. The culture fluid was centrifuged at 3000 rpm for 30 min. and the supernatant used for purification. The galactanase was purified from 700ml of the culture supernatant. The broth was diluted 1:1 with tap water and flocculated with Na-aluminate, C521 and A130. Filter aid was added and the suspension was filtered through a Propex23 filtration cloth giving a clear filtrate, which was further filtrated though a Seitz EKS filter plate (germ filtrate). The germ filtrate was concentrated by ultrafiltration on a 3 kDa cut-off polyethersulfone cassette and the concentrated enzyme was transferred to 10 mM Tris/HCl, pH 7.5 on a Sephadex G25 column. The G25 filtrate was applied to a 100 ml Q-sepharose FF column equilibrated in 10 mM Tris/HCl, pH 7.5. After washing the column with equilibration buffer, the galactanase was eluted with a linear NaCl gradient (0→0.5 M). Galactanase containing fractions were pooled and (NH₄)₂SO₄ was added to 1.6 final concentration. The enzyme was applied to a 100 ml Butyl Toyopearl 650S column equilibrated in 100 mM H₃BO₃, 10 mM Dimethyl glutaric acid, 2 mM CaCl₂, 1.6 M (NH₄)₂SO₄, pH 6.5. After washing the column, the galactanase was eluted with a linear (NH₄)₂SO4 gradient (1.6→OM). This gradient did not elute the galactanase and hence the enzyme was step eluted with 100 mM H₃BO₃, 10 mM Dimethyl glutaric acid, 2 mM CaCl₂, pH 6.5 with 25% (v/v) isopropanol added. Galactanase containing fractions were pooled and the pH was adjusted to pH 4.5 and the pool was diluted with deionised water to give the same conductivity as 20 mM CH₃COOH/NaOH, pH 4.5. The galactanase was applied to a 50 ml S-sepharose HP column equilibrated in 20 mM CH₃COOH/NaOH, pH 4.5. After washing the column with equilibration buffer, the galactanase was eluted with a linear NaCl gradient (0→0.5 M). Galactanase containing fractions were pooled and concentrated on an Amicon ultrafiltration cell (10 kDa cut-off membrane).

Finally, the concentrated enzyme preparation was applied to a Superdex200 size exclusion column equilibrated in 20 mM succinic acid/NaOH, 150 mM NaCl, pH 6.0. The galactanase was eluted with the same buffer and galactanase containing fractions were analysed by SDS-PAGE and almost pure fractions were pooled. The product (1999-01373-01) was freezed (−20° C.) in aliquots.

### EXAMPLE 7

Characterization of Bacillus pumilus Galactanase

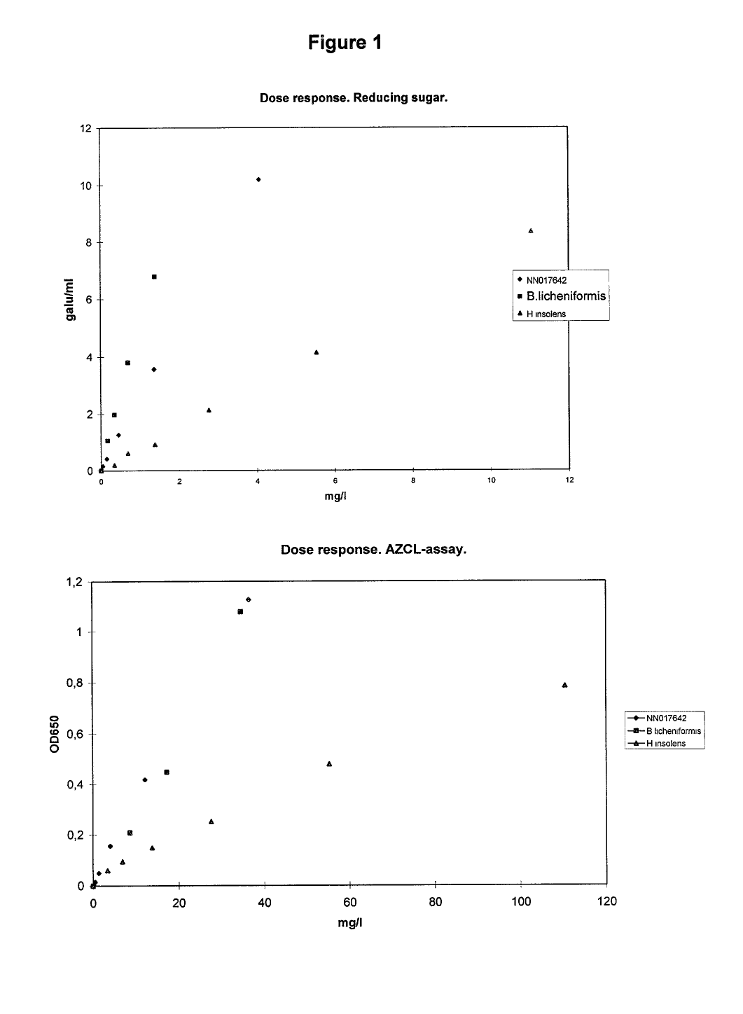

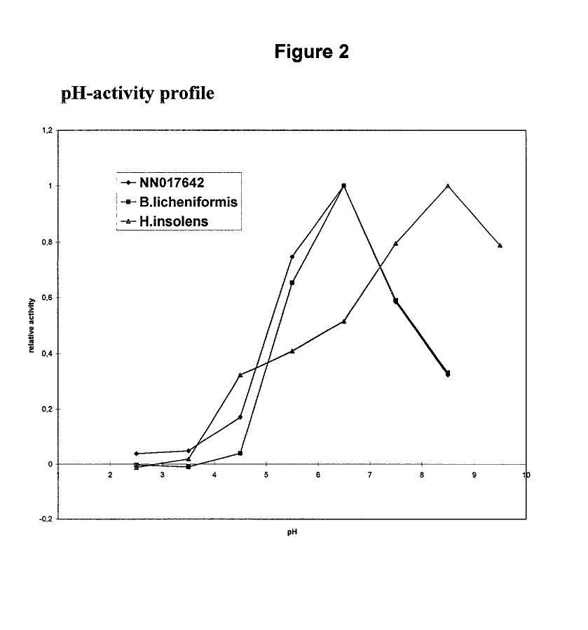

FIGS. 1 and 2 show dosis-response curves for the B. pumilus NN017642 galactanase and two other galactanases, isolated from B. licheniformis (WO 00/47711) and Humicola insolens (WO 97/32014), in the two different galactanase assays: Reducing sugar assay (FIG. 1) and AZCL assay (FIG. 2). Assay conditions were standard assay conditions, i.e. 37° C. and pH 6.5. The Reducing sugar assay was used for determination of specific activities and the AZCL assay was used for obtaining the pH-activity, pH-stability, and temperature-activity profiles (only the linear part of the AZCL dosis-response curves were used for this characterisation).

The specific activities shown in table 4 were determined as the ratio between the Reducing sugar assay (galu/ml) and the BCA protein determination (mg/ml). The Reducing sugar assay was performed at standard conditions (37° C., pH 6.5) and the activities were calculated from the linear part of the dosis-response curves.

TABLE 4

| | Activity | Protein | Spec. act. |
|---|---|---|---|
| Galactanase | (galu/ml) | (mg/ml) | (galu/mg) |
| B. pumilus NN017642 | 97.9 | 0.0366 | 2674 |
| B. licheniformis | 31936 | 5.55 | 5754 |
| H. insolens | 1611 | 2.21 | 729 |

It is seen from Table 4 that the B. pumilus NN017642 galactanase has a three to four times higher specific activity than the H. insolens galactanase.

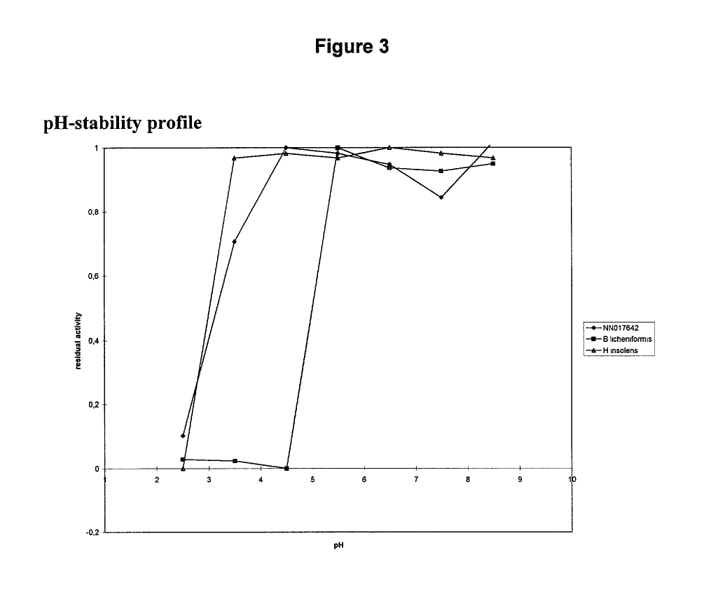

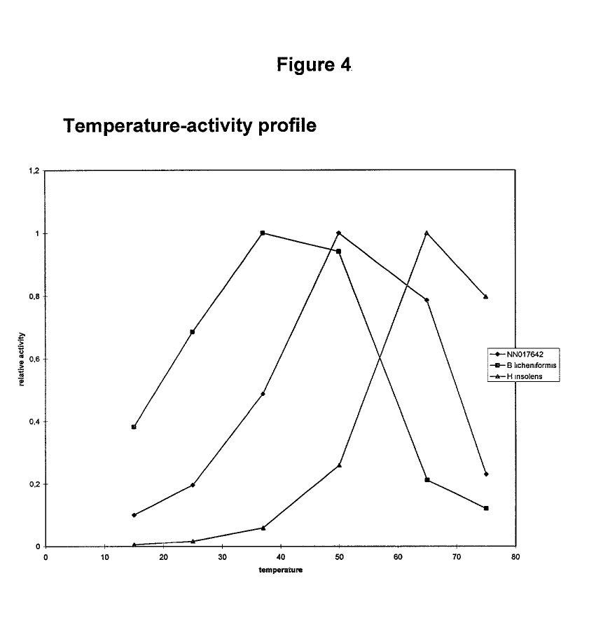

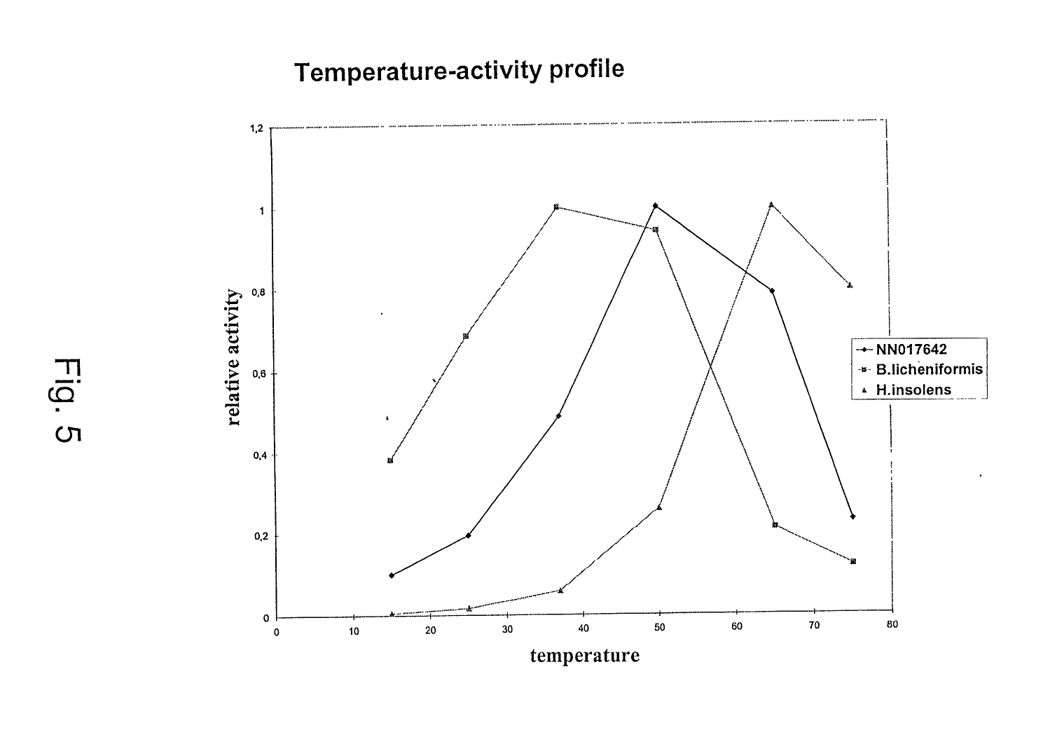

The pH-activity profile (FIG. 3) and the pH-stability profile (FIG. 4) of the B. pumilus, B. licheniformis, and H. insolens galactanases were determined at 37° C. at the indicated pH-values. The activities were plotted as relative activities—relative to the pH-value with highest activity. It is seen from FIG. 3 that the B. pumilus NN017642 galactanase has a pH-activity optimum almost as the B. licheniformis galactanase at a pH-value between 5.5 and 7.5 or between approx. 6 and 7.

The stability of the galactanases as a function of pH was determined after an incubation for two hours at 37° C. at the pH-values indicated in FIG. 4. The stability was plotted as residual stability (FIG. 4)—relative to samples kept for two hours in the refrigerator at a pH-value where the galactanase in question was stable. It is seen from FIG. 4 that the B. pumilus NNO17642 galactanase is much more stable at low pH-values than the B. licheniformis galactanase. It is therefore more likely that this galactanase could survive the stomach of a monogastric animal.

In FIG. 5 is shown the temperature activity profile of the galactanases. The galactanase activities were determined at pH 6.5 and at temperatures: 15° C., 25° C., 37° C., 50° C., 65° C., and 75° C. The activities were plotted as relative activities (FIG. 5)—relative to the temperature where the galactanase had the highest activity. It is seen from FIG. 5 that the B. pumilus NN017642 galactanase has a temperature-activity optimum at around 50° C., which is significantly higher than the B. licheniformis galactanase.

### EXAMPLE 8

Galactan Action Pattern Analysis

A comparison of the degradation mechanism of the two galactanases from Bacillus licheniformis and B. pumilus with a fungal galactanase from Myceliophthora thermophila (WO 97/32014) was carried out.

Enzyme digests were done as follows: 1% (w/v) lupin galactan (cat. no. P-GALLU from Megazyme) in 50 mM CH₃COOH, 50 mM KH₂PO₄, 50 mM H₃BO₃, 1 mM CaCl₂, 0.01% Triton X-100, pH 6.5, was digested at 35° C. with the three different galactanases, each dosed with the same amount of activity and containing typically 1 pmol galactanase per ml reaction mixture. The enzymatic reaction was initiated by addition of 300 μl enzyme solution in buffer to 600 μl 1.5% (w/v) lupin galactan solution. At suitable time intervals the hydrolysis products were analysed.

The degradation products from galactan were identified and quantified by HPLC on a Dionex system using a PA100 column from Dionex. Sample aliquots of 10 μl were applied to the column and the oligosaccharides were eluted using a gradient from 0.1 M NaOH (Buffer A) to 50 mM NaOH, 0.5 M NaOAc (Buffer B) at a flow of 1 ml/min. The oligosaccharides were detected and quantified using pulsed amperometry (ED40 from Dionex). An overview of the results is seen as Table 5.

TABLE 5
Galactan Action Pattern. The values in the table are the peak
areas. A peak area is the area under the μC signal from the ED40
detector.

| | Reaction | DP1 | DP2 | DP3 | DP4 | DP5-10 |
|---|---|---|---|---|---|---|
| | time | (peak | (peak | (peak | (peak | (peak |
| Galactanase | (min) | area) | area) | area) | area) | area) |
| Bacillus | 0 | nd | 1477 | nd | 86 | nd |
| licheniformis | 41 | 702 | 1972 | 432 | 1481 | 3126 |
| | 82 | 736 | 2075 | 751 | 3114 | 5523 |
| | 124 | 768 | 2134 | 1069 | 5423 | 6207 |
| | 287 | 1047 | 2840 | 2614 | 14274 | 14910 |
| | 451 | 1306 | 3622 | 4231 | 22131 | 20679 |
| | 615 | 1607 | 4420 | 5783 | 29085 | 26174 |
| | 779 | 1717 | 5064 | 7005 | 32018 | 27042 |
| Bacillus | 0 | 761 | 550 | 95 | 58 | nd |
| pumilus | 42 | 1064 | 1160 | 746 | 3688 | 936 |
| | 83 | 613 | 1506 | 1262 | 6813 | 936 |
| | 124 | 647 | 2374 | 2671 | 10107 | 1815 |
| | 216 | 874 | 1873 | 2752 | 17773 | 2330 |
| | 380 | 1350 | 2620 | 4597 | 25544 | 5496 |
| | 707 | 1671 | 2618 | 8296 | 38700 | 8087 |
| | 871 | 1719 | 2150 | 9419 | 42006 | 20196 |
| Myceliophtora | 0 | 931 | 2144 | 389 | 55 | 965 |
| thermophila | 41 | 1160 | 2161 | 288 | 240 | 1192 |
| | 82 | 1386 | 2215 | 450 | 490 | 3703 |
| | 124 | 1585 | 2267 | 647 | 804 | 5782 |
| | 288 | 2329 | 2735 | 1644 | 2287 | 16459 |
| | 451 | 3041 | 3361 | 2771 | 4626 | 29306 |
| | 615 | 3865 | 5000 | 4854 | 6993 | 40997 |
| | 779 | 4842 | 5884 | 6135 | 7262 | 49705 |

It is seen from the Table 5 that the two Bacillus galactanases release mainly DP4 oligogalactans, whereas the M. thermophila galactanase releases a mixture of oligogalactans. This clearly shows that the Bacillus galactanases have a different action pattern on lupin galactan than the M. thermophila enzyme.

In summary, our experiments show that the Bacillus galactanases are DP4-galactanases.

### EXAMPLE 9

Recombinant Expression of the Galactanase from B. pumilus

Media

TY (as described in Ausubel, F. M. et al. (eds.) “Current protocols in Molecular Biology”. John Wiley and Sons, 1995).

LB agar (as described in Ausubel, F. M. et al. (eds.) “Current protocols in Molecular Biology”. John Wiley and Sons, 1995).

LBPG is LB agar supplemented with 0.5% Glucose and 0.05 M potassium phosphate, pH 7.0

AZCL-galactan is added to LBPG-agar to 0.5%, AZCL-galactan is from Megazyme, Ireland.

BPX media is described in EP 0 506 780 (WO 91/09129).

Strains

B. subtilis strains: DN1885 (Diderichsen, B., Wedsted, U., Hedegaard, L., Jensen, B. R., SjØoholm, C. (1990) Cloning of aldB, which encodes alpha-acetolactate decarboxylase, an exoenzyme from Bacillus brevis. J. Bacteriol., 172, 4315-4321). PL1801 (B. subtilis DN1885 where the two major proteases have been inactivated).

Competent cells were prepared and transformed as described by Yasbin, R. E., Wilson, G. A. and Young, F. E. (1975) Transformation and transfection in lysogenic strains of Bacillus subtilis: Evidence for selective induction of prophage in competent cells. J. Bacteriol, 121:296-304.

Plasmids

pMOL944: This plasmid is a pUB110 derivative essentially containing elements making the plasmid propagatable in Bacillus subtilis, kanamycin resistance gene and having a strong promoter and signal peptide cloned from the amyL gene of B. licheniformis ATCC14580. The signal peptide contains a SacII and a PstI site making it convenient to clone the DNA encoding the mature part of a protein in-fusion with the signal peptide. This results in the expression of a Pre-protein which is directed towards the exterior of the cell.

Construction of pMOL944: The pUB110 plasmid (McKenzie, T. et al., 1986, Plasmid 15:93-103) was digested with the unique restriction enzyme NciI. A PCR fragment amplified from the amyL promoter encoded on the plasmid pDN1981 (P. L. JØrgensen et al.,1990, Gene, 96, p37-41.) was digested with NciI and inserted in the NciI digested pUB110 to give the plasmid pSJ2624. The two PCR primers used have the following sequences:

#LWN5494 (SEQ ID NO:4):
5′-GTCGCCGGGGCGGCCGCTATCAATTGGTAACTGTATCTCAGC
#LWN5495 (SEQ ID NO:5):
5′-GTCGCCCGGGAGCTCTGATCAGGTACCAAGCTTGTCGACCTGCAGAATGAGGCAGCAAGAAGAT

The primer #LWN5494 (SEQ ID NO: 4) inserts a NotI site in the plasmid. The plasmid pSJ2624 was then digested with SacI and NotI and a new PCR fragment amplified on amyL promoter encoded on the pDN1981 was digested with SacI and NotI and this DNA fragment was inserted in the SacI-NotI digested pSJ2624 to give the plasmid pSJ2670.

This cloning replaces the first amyL promoter cloning with the same promoter but in the opposite direction. The two primers used for PCR amplification have the following sequences:

#LWN5938 (SEQ ID NO:6)
5′-GTCGGCGGCCGCTGATCACGTACCAAGCTTGTCGACCTGCAGAATGAGGCAGCAAGAAGAT
#LWN5939 (SEQ ID NO:7):
5′-GTCGGAGCTCTATCAATTGGTAACTGTATCTCAGC

The plasmid pSJ2670 was digested with the restriction enzymes PstI and BclI and a PCR fragment amplified from a cloned DNA sequence encoding the alkaline amylase SP722 (disclosed in International Patent Application published as WO 95/26397 which is hereby incorporated by reference in its entirety) was digested with PstI and BclI and inserted to give the plasmid pMOL944. The two primers used for PCR amplification have the following sequence:

#LWN7864 (SEQ ID NO:8):
5′-AACAGCTGATCACGACTGATCTTTTAGCTTGGCAC-3′
#LWN7901 (SEQ ID NO:9):
5′-AACTGCAGCCGCGGCACATCATAATGGGACAAATGGG-3′

The primer #LWN7901 (SEQ ID NO: 9) inserts a SacII site in the plasmid.

pPL3143: This plasmid is a pMOL944 derivative in which a terminator has been inserted between the SacII and the NotI site in pMOL944. At the same time a new restriction site for cloning AscI has been inserted. The plasmid was constructed by means of ordinary genetic engineering and is briefly described in the following.

Construction of pPL3143: The plasmid pMOL944 was digested with SacII and NotI. A PCR fragment generating a terminator was made using the two primers listed below and plasmid pMOL944 as template. This fragment was digested with EagI and SacII and inserted between the SacII and the NotI site in PMOL944 to create the plasmid pPL3143.

Primer 130721 (SEQ ID NO:10):
5′-CGATCGGCCGATAAAAAAACCGGGCGGAAACCGCCCGTCATCTGGCGCGCCTATATACCGCGGCTGCAGAATGAGGCAGCAAG-3′
Primer 130722 (SEQ ID NO:11):
5′-GGCGCATTAACGGAATAAAGGGTGT-3′

The galactanase encoding DNA sequence was PCR amplified using the PCR primer set consisting of these following two oligo nucleotides, restriction sites PstI and AscI are underlined:

Galac .upper.PstI (SEQ ID NO:12):
5′-GCCTCTGCAGCCGCGGCGGATGCAATTTCAGTTCAGCCG-3′
Galac .lower.AscI (SEQ ID NO:13):
5′GCGTGGCGCGCCTTACGGTGTCATTTGTTTAAATAC-3′

Plasmid DNA from E. coli strain DSM 13286 was used as template in a PCR reaction using Amplitaq DNA Polymerase (Perkin Elmer) according to manufacturers instructions. The PCR reaction was set up in PCR buffer (10 mM Tris-HCl, pH 8.3, 50 mM KCl, 1.5 mM MgCl₂, 0.01% (w/v) gelatin) containing 200 μM of each dNTP, 2.5 units of AmpliTaq polymerase (Perkin-Elmer, Cetus, USA) and 100 pmol of each primer

The PCR reaction was performed using a DNA thermal cycler (Landgraf, Germany). One incubation at 94° C. for 1 min followed by thirty cycles of PCR performed using a cycle profile of denaturation at 94° C. for 30 sec, annealing at 60° C. for 1 min, and extension at 72° C. for 2 min. Five-μl aliquots of the amplification product was analysed by electrophoresis in 0.7% agarose gels (NuSieve, FMC). The appearance of a DNA fragment with a size of approx. 1.2 kb indicated proper amplification of the gene segment.

Subcloning of PCR fragment

Forty five μl aliquots of the PCR products generated as described above were purified using QIAquick PCR purification kit (Qiagen, USA) according to the manufacturer's instructions. The purified DNA was eluted in 50 μl of 10 mM Tris-HCl, pH 8.5.

5 μg of pPL3143 and twenty five μl of the purified PCR fragment was digested with PstI and AscI, electrophoresed in 0.8 % low gelling temperature agarose (SeaPlaque GTG, FMC) gels, the relevant fragments were excised from the gels, and purified using QIAquick Gel extraction Kit (Qiagen, USA) according to the manufacturer's instructions. The isolated PCR DNA fragment was then ligated to the PstI-AscI digested and purified pPL3143. The ligation was performed overnight at 16° C. using 0.5 μg of each DNA fragment, 1 U of T4 DNA ligase and T4 ligase buffer (Boehringer Mannheim, Germany).

The ligation mixture was used to transform competent B. subtilis PL1801 cells. The transformed cells were plated onto LBPG agar plates containing 10 ug/ml of Kanamycin and 0,2% AZCL-Galactan (Megazyme). After 18 hours incubation at 37° C. colonies were seen on plates. Several clones showing a blue halo around the colony were analyzed by isolating plasmid DNA from overnight culture broth.

One such positive clone was restreaked several times on agar plates as used above, this clone was called PL3372. The clone B. subtilis PL3372 was grown overnight in TY-10μg/ml Kanamycin at 37° C., and next day 1 ml of cells were used to isolate plasmid from the cells using the Qiaprep Spin Plasmid Miniprep Kit #27106 according to the manufacturers recommendations for B. subtilis plasmid preparations. This DNA was DNA sequenced and revealed the DNA sequence corresponding to the mature part of the Bacillus pumilus galactanase shown in SEQ ID NO. 1.

### EXAMPLE 10

Purification of Bacillus pumilus Galactanase Expressed in Bacillus subtilis

Expression of the B. pumilus galactanase in B. subtilis was performed by growing PL3372 in 200 ml BPX media supplemented with 10 μg/ml Kanamycin in 500 ml baffled shake flasks for 5 days at 37° C. at 300 rpm. The culture broth was diluted 1:1 with water and flocculated with Na-aluminate, C521 and A130. Filter aid was added and the suspension was filtered through a Propex23 filtration cloth giving a clear filtrate, which was further filtrated though a Seitz EKS filter plate (germ filtrate). The germ filtrate was concentrated by ultrafiltration on a 3kDa cut-off polyethersulfone cassette and the concentrated enzyme was transferred to 10 mM Tris/HCl, pH 7.5 on a Sephadex G25 column. The G25 filtrate was applied to a 100 ml Q-sepharose FF column equilibrated in 10 mM Tris/HCl, pH 7.5. After washing the column with equilibration buffer, the galactanase was eluted with a linear NaCl gradient (0 to 0.5M).

Galactanase containing fractions were pooled and (NH₄)₂SO₄ was added to 1.5 M final concentration. The enzyme was applied to a 100 ml Butyl Toyopearl 650S column equilibrated in 100 mM H₃BO₃, 10 mM dimethyl glutaric acid, 2 mM CaCl₂, 1.5 M (NH₄)₂SO₄, pH 6.5. After washing the column, the galactanase was eluted with a linear gradient between the equilibration buffer and 100 mM H₃BO₃, 10 mM Dimethyl glutaric acid, 2 mM CaCl₂, 25% (v/v) isopropanol, pH 6.5.

Galactanase containing fractions were pooled and the enzyme was transferred to 20 mM CH₃COOH/NaOH, pH 4.5 on a Sephadex G25 column. The galactanase was applied to a 100 ml S-sepharose HP column equilibrated in 20 mM CH₃COOH/NaOH, pH 4.5. After washing the column with equilibration buffer, the galactanase was eluted with a linear NaCl gradient (0 to 0.5M).

Galactanase containing fractions were analysed by SDS-PAGE and pure fractions were pooled. The product (1999-10209-01) was freezed (−20° C.) in aliquots. The N-terminal sequence for the purified recombinant B. pumilus galactanase (product 1999-10209-01) was: ADAISVQPINGLQGDFIKGADI. This N-terminal sequence is identical to the sequence obtained for wild-type B. pumilus galactanase, except for the first amino acid residue (product 1999-01373-01 sequence: DAISVQPINGLQGDFIKGADISMLAE). In addition, the two products had the same molecular weight (Mᵣ) when analyzed by SDS-PAGE, indicating that except for the first amino acid residue the two products are identical.

### EXAMPLE 11

Testing of Galactanase Derived from Bacillus pumilus in an in Vitro Digestion System

The galactanase derived from Bacillus pumilus is tested for its ability to release D-galactose from a maize-SBM (maize-Soy Bean Meal) substrate in an in vitro digestion system (simulating digestion in monogastric animals). For the blank treatments, maize-SBM is incubated in the absence of exogenous galactanase. Additive and/or synergistic effects of combinations of B. pumilus galactanase with other animal feed enzymes, in particular carbohydrases such as lactase may be tested as well.

The in vitro system consists of 30 flasks in which maize-SBM substrate is initially incubated with HCl/pepsin—simulating gastric digestion—and subsequently with pancreatin—simulating intestinal digestion. At the end of the gastric incubation period samples of in vitro digesta are removed and analysed for released D-galactose.

Substrates

10 g maize-SBM diet with a maize-SBM ratio of 6:4 (w/w) is used.

Digestive enzymes

Pepsin (Sigma P-7000; 539 U/mg, solid), pancreatin (Sigma P-7545); 8×U.S.P. (US Pharmacopeia).

Schematic outline of in vitro digestion procedure

| | | | | Simulated |
|---|---|---|---|---|
| | | | Time | digestion |
| Components added to flask | pH | Temp. | (min) | phase |
| 10 g maize-SBM diet (6:4), | 3.0 | 40° C. | t = 0 | Gastric |
| HCl/pepsin (3000 U/g | | | | |
| diet), B.p. galactanase | | | | |
| (0.1 mg enzyme protein/g | | | | |
| diet) in presence or | | | | |
| absence of other animal | | | | |
| feed enzymes, in | | | | |
| particular carbohydrases | | | | |
| such as lactase | | | | |
| NaOH | 6.8 | 40° C. | t = 60 | Intestinal |
| NaHCO₃/pancreatin (8 mg/g | 6.8 | 40° C. | t = 90 | |
| diet) | | | | |
| Stop incubation, remove | 7.0 | 0° C. | t = 330 | |
| aliquot | | | | |

Experimental Procedure for in Vitro Model

1) 10 g of substrate is weighed into a 100 ml flask.

2) At time 0 min, 46 ml HCl (0.1 M) containing pepsin (3000 U/g diet) and 1 ml of enzyme solution (0.1 mg enzyme protein/g diet) is added to the flask while mixing. The flask is incubated at 40° C.

3) At time 30 min, pH is measured.

4) At time 45 min, 16 ml of H₂O is added.

5) At time 60 min, 7 ml of NaOH (0.39 M) is added.

6) At time 90 min, 5 ml of NaHCO₃ (1M) containing pancreatin (8.0 mg/g diet) is added.

7) At time 120 min, pH is measured.

8) At time 300 min, pH is measured.

9) At time 330 min, samples of 30 ml are removed and placed on ice before centrifugation (10000×g, 10 min, 4° C.).

10) Supernatants are removed and stored at -20° C.

Testing of intestinal galactanase performance

As a variation of the experimental procedure described above, the B.p. galactanase or enzyme combinations containing B.p. galactanase may be first added after completion of step 6. Thus, enzyme performance is tested under conditions which imply a 100% survival of enzyme activity during exposition to low pH in the preceding stomach-like digestion (steps 1-4).

D-galactose Determinations

Determination of D-galactose in in-vitro digesta is carried out by using a photometric assay (for example lactose/D-galactose kit from Boehringer Mannheim, Germany) as described by Kurz, G. and Wallenfels, K. (1974) in Methods of Enzymatic Analysis (Bergmeyer, H.U., ed.) 2nd ed., vol.3, pp. 1180-1184 and 1279-1282, Verlag Chemie, Weinheim/Academic Press Inc., New York and London.

1. An isolated polypeptide having galactanase activity comprising an amino acid sequence: Nxx(M/L)FDFxGxxLxS, of SEQ ID NO: 1: wherein x may be any amino acid and (M/L) is M or L, and wherein the polypeptide maintains at least 30% residual galactanase activity after two hours at a temperature of 37° C. at pH 4.5.

2. The isolated polypeptide of claim 1, wherein the polypeptide is isolated from a Bacillus cell.

3. The isolated polypeptide of claim 1, which has an activity optimum at a pH between 5.5 and 7.5.

4. The isolated polypeptide of claim 3, which has an activity optimum at a pH between 6 and 7.

5. The isolated polypeptide of claim 1, which has an optimum activity at a temperature between 30° C. and 70° C.

6. The isolated polypeptide of claim 1, which mainly releases DP4 oligogalactans when degrading galactan.

7. An isolated polypeptide having galactanase activity comprising an amino acid sequence of amino acid residues 1 to 402 of SEQ ID NO: 3 or an amino acid sequence that is at least 70% identical to the amino acid sequence of amino acid residues 1 to 402 of SEQ ID NO: 3.

8. The isolated polypeptide of claim 7, wherein the polypeptide is isolated from a Bacillus cell.

9. The isolated polypeptide of claim 7, which has an activity optimum at a pH between 5.5 and 7.5.

10. The isolated polypeptide of claim 9, which has an activity optimum at a pH between 6 and 7.

11. The isolated polypeptide of claim 7, which has an optimum activity at a temperature between 30° C. and 70° C.

12. The isolated polypeptide of claim 7, wherein the polypeptide maintains at least 30% residual galactanase activity after two hours at a temperature of 37° C. at pH 4.5.

13. The isolated polypeptide of claim 7, which mainly releases DP4 oligogalactans when degrading galactan.

14. An isolated polynucleotide molecule encoding a polypeptide of claim 1, comprising a polynucleotide sequence of nucleotides 1 to 1209 of SEQ ID NO: 2 or a polynucleotide sequence that is at least 70% identical to the polynucleotide sequence of nucleotides 1 to 1209 of SEQ ID NO: 2.

15. The isolated polynucleotide molecule of claim 14, wherein the polynucleotide is DNA.

16. An expression vector comprising a DNA segment selected from the group consisting of:
a) DNA molecules encoding a polypeptide having galactanase activity comprising a nucleotide sequence of nucleotides 1 to 1209 of SEQ ID NO: 2,
b) DNA molecules encoding a polypeptide having galactanase activity that is at least 70% identical to the amino acid sequence of amino acid residues 1 to 402 of SEQ ID NO: 3,
c) DNA molecules encoding a galactanase enzyme comprising the amino acid sequence: Nxx(M/L)FDFxGxxLxS of SEQ ID NO: 1, wherein x is any amino acid and (M/L) is M or L, and wherein the galactanase maintains at least 30% residual galactanase activity after two hours at a temperature of 37° C. at pH 4.5; and
d) degenerate DNA sequences of (a) (b) or (c).

17. A cell into which has been introduced an expression vector of claim 16, wherein said cell expresses the polypeptide encoded by the polynucleotide sequence.

18. A method of producing a polypeptide having galactanase activity comprising culturing a cell of claim 17; and recovering the polypeptide.

19. An enzyme preparation comprising an isolated polypeptide of claim 1.

20. The preparation of claim 19, which further comprises one or more enzymes selected from the group consisting of proteases, carbohydrases, lipases, reductases, oxidases, transglutaminases, and phytases; and mixtures thereof.

21. An enzyme preparation comprising an isolated polypeptide of claim 7.

22. The preparation of claim 21, which further comprises one or more enzymes selected from the group consisting of proteases, carbohydrases, lipases, reductases, oxidases, transglutaminases, and phytases; and mixtures thereof.

23. A method for modifying and/or improving the nutritional value of an animal feed, the method comprising adding to the animal feed at least one galactanase enzyme selected from the group consisting of:
a) a galactanase enzyme comprising an amino acid sequence of SEQ ID NO: 3;
b) an analogue of the enzyme defined in a), said analogue comprising an amino acid sequence which is at least 70% identical to the sequence of SEQ ID NO: 3, or which can be derived from said sequence by substitution, deletion or addition of at least one amino acid; and
c) a galactanase enzyme comprising the amino acid sequence: Nxx(M/L)FDFxGxxLxS of SEQ ID NO: 1, wherein x is any amino acid and (M/L) is M or L, and wherein said galactanase maintains at least 30% residual galactanase activity after two hours at a temperature of 37° C. at pH 4.5.

24. The method of claim 23, wherein the galactanase is an analogue of the galactanase enzyme defined in a), wherein said analogue has an amino acid sequence that is at least 70% identical with the galactanase enzyme in a).

25. The method of claim 24, wherein the galactanase is an analogue of the galactanase enzyme defined in a), wherein said analogue has an amino acid sequence that is at least 80% identical with the galactanase enzyme in a).

26. The method of claim 25, wherein the galactanase is an analogue of the galactanase enzyme defined in a), wherein said analogue has an amino acid sequence that is at least 90% identical with the galactanase enzyme in a).

27. The method of claim 26, wherein the galactanase is an analogue of the galactanase enzyme defined in a), wherein said analogue has an amino acid sequence that is at least 95% identical with the galactanase enzyme in a).

28. The method of claim 23, wherein the animal feed comprises plant material.

29. The method of claim 28, wherein the plant material comprises soybean (Glycine max).

30. The method of claim 23, wherein the galactanase enzyme is of bacterial origin.

31. The method of claim 30, wherein the galactanase enzyme is derived from a strain of Bacillus.

32. The method of claim 23, further comprising adding to the animal feed one or more enzymes selected from the group consisting of proteases, carbohydrases, lipases, reductases, oxidases, transglutaminases, and phytases; and mixtures thereof.

33. A method for modifying and/or improving the nutritional value of an animal feed, comprising adding to the animal feed at least one galactanase enzyme comprising an amino acid sequence encoded by a DNA sequence which hybridizes to the DNA sequence of SEQ ID NO: 2 under medium stringency conditions.

34. The method of claim 33, wherein the galactanase enzyme comprises an amino acid sequence encoded by a DNA sequence which hybridizes to the DNA sequence of SEQ ID NO: 2 under high stringency conditions.

35. The method of claim 33, wherein the animal feed comprises plant material.

36. The method of claim 35, wherein the plant material comprises soybean (Glycine max).

37. The method of claim 33, further comprising adding to the animal feed one or more enzymes selected from the group consisting of proteases, carbohydrases, lipases, reductases, oxidases, transglutaminases, and phytases; and mixtures thereof.

38. A method for obtaining a DNA sequence encoding a galactanase enzyme or a portion thereof, wherein said DNA sequence is detected using a probe comprising a DNA subsequence of the DNA sequence of SEQ ID NO: 2 or of a DNA sequence complementary to the DNA sequence of SEQ ID NO: 2, said subsequence comprising at least 16 nucleotides, or preferably at least 18 nucleotides.

39. A method for obtaining a DNA sequence encoding a galactanase enzyme or a portion thereof, wherein said DNA sequence is detected using a probe comprising an oligonucloetide sequence of at least 16 nucleotides, which hybridizes to the DNA sequence of SEQ ID NO: 2, or to the complementary DNA sequence, under medium stringency conditions.

40. An animal feed additive comprising a galactanase of claim 1.

41. An animal feed additive comprising a galactanase of claim 7.

42. An animal feed composition comprising a galactanase of claim 1.

43. An animal feed composition comprising a galactanase of claim 7.


SEQUENCE LISTING (13 sequences)

SEQ ID NO: 1 - protein, 14 aa, Bacillus
  misc_feature (2)..(2): Xaa  denotes any amino acid.
NXXXFDFXGXXLXS

SEQ ID NO: 2 - DNA, 1209 bp, Bacillus pumilus NN017642
  CDS (1)..(1206)
atgaagaaatggatgcttgtcacagttgtcgttttctttgtgatggggttaaggattttacccgaagcaaaagcggatgcaatttcagttcagccgatcaatggattgcaaggtgatttcattaaaggagcagatatctccatgcttgcggaagtggagagaagcgggggcagatacttcgaccagaatggaaaacaggtggacccgctcaagtttttaaaagaaaaaggcgtcaactatgtgcgtattagactgtggaaccacccatacgacaaccaaggtcgtgcctataatggcgggacaaatgatctcaatacagcgatcgctttatcgaaacgtgcaaaagcacaaaatatgaaagtactgcttgattttcactacagtgatttctggaccgatcctggtaaacagttcaaaccgaaagcatgggcgtccctttcacaaaacgatttagtaaaagcggttggcacttacacaggggatgtactcaaaaccatgcgagcacaaaatgctttgccgaacatggtacaggtcggaaatgaattgaactcaggcatgctgtggccgaacggcaaaagctggggtgaaggcggcggtgaatttgaccggcttgccgctttgctaaaggctggcacaaatgctgttcgctctgtcgattcaaatatcaacatcatgcttcacctagcccatggtggggacaacggggcctcccgctggtggtttgatgaaatcaccaaacgaggcgtttcgttcgatacgatcggtttatcttactacccgtattgggacggaggatttagcggtcttactaacaatatgaatgatattagcgcccgctatcacaaagatgtcatcgtggtagaaaccgcctacggctttacaacagcaaatggtgacaacctagataactcatttaatcaagattcagtcaatactgccggttatccagcatcgccacaagggcaggcatcttttatacgagatctatcagagaaaatttcacaagtaaaaagcaatcgtgggaaagggttcttttattgggagccgctttggattccagcaaaaggtgcaccgtggtctagccaatacgggctcgcttatatccaaacaaccggtacagtcggaaatgcttgggaaaatcaggccatgtttgatttcaacggaaaagcactgccttctcttgatgtatttaaacaaatgacaccgtaa

SEQ ID NO: 3 - protein, 402 aa, Bacillus pumilus NN017642
MKKWMLVTVVVFFVMGLRILPEAKADAISVQPINGLQGDFIKGADISMLAEVERSGGRYFDQNGKQVDPLKFLKEKGVNYVRIRLWNHPYDNQGRAYNGGTNDLNTAIALSKRAKAQNMKVLLDFHYSDFWTDPGKQFKPKAWASLSQNDLVKAVGTYTGDVLKTMRAQNALPNMVQVGNELNSGMLWPNGKSWGEGGGEFDRLAALLKAGTNAVRSVDSNINIMLHLAHGGDNGASRWWFDEITKRGVSFDTIGLSYYPYWDGGFSGLTNNMNDISARYHKDVIVVETAYGFTTANGDNLDNSFNQDSVNTAGYPASPQGQASFIRDLSEKISQVKSNRGKGFFYWEPLWIPAKGAPWSSQYGLAYIQTTGTVGNAWENQAMFDFNGKALPSLDVFKQMTP

SEQ ID NO: 4 - DNA, 42 bp, Artificial Sequence
  Primer
gtcgccggggcggccgctatcaattggtaactgtatctcagc

SEQ ID NO: 5 - DNA, 64 bp, Artificial Sequence
  Primer
gtcgcccgggagctctgatcaggtaccaagcttgtcgacctgcagaatgaggcagcaagaagat

SEQ ID NO: 6 - DNA, 61 bp, Artificial Sequence
  Primer
gtcggcggccgctgatcacgtaccaagcttgtcgacctgcagaatgaggcagcaagaagat

SEQ ID NO: 7 - DNA, 35 bp, Artificial Sequence
  Primer
gtcggagctctatcaattggtaactgtatctcagc

SEQ ID NO: 8 - DNA, 35 bp, Artificial Sequence
  Primer
aacagctgatcacgactgatcttttagcttggcac

SEQ ID NO: 9 - DNA, 37 bp, Artificial Sequence
  Primer
aactgcagccgcggcacatcataatgggacaaatggg

SEQ ID NO: 10 - DNA, 83 bp, Artificial Sequence
  Primer
cgatcggccgataaaaaaaccgggcggaaaccgcccgtcatctggcgcgcctatataccgcggctgcagaatgaggcagcaag

SEQ ID NO: 11 - DNA, 25 bp, Artificial Sequence
  Primer
ggcgcattaacggaataaagggtgt

SEQ ID NO: 12 - DNA, 39 bp, Artificial Sequence
  Primer
gcctctgcagccgcggcggatgcaatttcagttcagccg

SEQ ID NO: 13 - DNA, 36 bp, Artificial Sequence
  Primer
gcgtggcgcgccttacggtgtcatttgtttaaatac